(19) United States
(12) Patent Application Publication
(10) Pub. No.: US 2011/0306046 A1
(43) Pub. Date: Dec. 15, 2011

(54) Probes and Primers for Detection of Malaria

(75) Inventors: Manjula Jagannath, Karnataka (IN); Chandrasekhar Bhaskaran Nair, Karnataka (IN); Pillarisetti Venkata Subbarao, Karnataka (IN); Mulakkapurath Narayanan Manoj, Karnataka (IN); Shamamandri Markandaiah Shashirekha, Karnataka (IN)

(73) Assignee: BigTec Private Limited, Bangalore, Karnataka (IN)

(21) Appl. No.: 13/202,842

(22) PCT Filed: Jan. 27, 2010
(86) PCT No.: PCT/IN2010/000049

§ 371 Date: Aug. 23, 2011

(30) Foreign Application Priority Data
Feb. 24, 2009 (IN) 00421/CHE/2009

(51) Int. Cl. C12Q 1/68; C07H 21/04

(52) U.S. Cl. 435/6.11; 536/24.32; 536/24.33

(57) ABSTRACT

The present disclosure gives description of a method used for the detection and quantification of malarial infection caused either by Plasmodium falciparum or Plasmodium vivax using nucleic acids isolated from blood samples by employing Oligonucleotide probes. The method employed here for detection is by Real time PCR.

### FIELD OF THE DISCLOSURE

The present disclosure is in relation to a method for the detection and quantification of malarial infection caused either by Plasmodium falciparum or Plasmodium vivax. The method makes use of nucleic acids isolated from blood samples by employing “Oligonucleotide” probes.

### BACKGROUND AND PRIOR ARTS OF THE DISCLOSURE

Malaria remains an urgent problem in global public health with the annual death toll of 0.7-2.7 million, with more than 75% of the victims being African children. Over the past 35 years, the incidence of malaria has increased 2-3 fold. At present it affects 300-500 million people and causes about one million deaths, primarily in Africa. In 1955, the World Health Organization (WHO) began an ambitious programme to eradicate malaria through clinical treatment using chloroquine and by control of the mosquito population using DDT (Dichlorodiphenyl trichloroethane). Phased out in the late 1960s, the programme nevertheless resulted in an important and sustained reduction in the burden of the disease in many countries throughout the world. However, in many countries there has been resurgence in malaria. This has resulted essentially from the emergence and spread of drug resistant parasites. The evolution of insecticide-resistant mosquitoes, increased population density (the world population has doubled since 1963), global warming (which has allowed the spread of vectors into areas that were previously outside their range), continuing poverty, political instability and loss of productivity due to infectious diseases all these factors undermine the maintenance of a stable public-health infrastructure for the treatment and control of malaria.

Human malaria is a parasitic disease that is endemic in most tropical and subtropical ecosystems worldwide. Malarial parasites belong to the genus Plasmodium and infect many vertebrate hosts, including several species of non-human primate. Four Plasmodium species are parasitic to humans: Plasmodium falciparum, P. malariae, P. ovale and P. vivax. Of these, P. falciparum and P. vivax are associated with most malaria morbidity and mortality, respectively.

For proper treatment of malaria patients, accurate and rapid diagnosis of malaria is essential. The microscopic examination of a blood smear is the “gold standard” for malaria diagnosis. The method is sensitive and specific but laborious and time consuming. Due to some of the limitations of light microscopy for diagnosing malaria at the primary health care level, alternative methods, such as PCR and rapid antigen capture assays have been developed.

### OBJECTIVES OF THE PRESENT DISCLOSURE

The objective of the present disclosure is to provide probes and primers for the detection of malaria.

Another objective of the present disclosure is to provide a PCR reaction mixture for the detection of malaria.

Yet another objective of the present disclosure is a method for the detection and quantification of the malarial infection.

Still another objective of the present disclosure is to provide a kit for the detection of the malarial infection.

### SUMMARY OF THE DISCLOSURE

Accordingly, the present disclosure relates to Probes having SEQ ID Nos. 1, 2, and 3; primers of SEQ ID Nos. 4 or 10, 5, 6, 7, 8, and 9; a PCR reaction mixture for detection of malaria, said mixture comprising the sample to be detected, nucleic acid amplification reagents, probes selected from a group comprising SEQ ID Nos. 1, 2, and 3 and corresponding primers selected from a group comprising SEQ ID Nos. 4 or 10, 5, 6, 7, 8, and 9; a method of detecting and optionally quantifying malarial infection, said method comprising steps of: a) forming a reaction mixture comprising a sample to be detected, nucleic acid amplification reagents, probes selected from a group comprising SEQ ID Nos. 1, 2, and 3 and corresponding primers selected from a group comprising SEQ ID Nos. 4 or 10, 5, 6, 7, 8, and 9, b) subjecting the reaction mixture to PCR to obtain copies of target sequence followed by measuring any increase in fluorescence signal for detecting the malarial infection and c) optionally constructing a standard curve from the detected signal to obtain copy number for quantifying the malarial infection; and a kit for detection of malarial infection, said kit comprising dual labeled probes of SEQ ID Nos. 1, 2, and 3 individually or in combination, corresponding pair of primers of SEQ ID Nos. 4 or 10, 5, 6, 7, 8, and 9 individually or in combination and amplification reagents.

### BRIEF DESCRIPTION OF THE ACCOMPANYING FIGURES

FIG. 1 shows a Plasmodium falciparum standard curve.

FIG. 2 shows a Plasmodium vivax standard curve.

### DETAILED DESCRIPTION OF THE DISCLOSURE

The present disclosure relates to probes having SEQ ID Nos. 1, 2, and 3.

In an embodiment of the present disclosure, said probes are for detection of malaria.

In another embodiment of the present disclosure, the probes are conjugated with detectable labels having fluorophore at 5′ end and a quencher in internal region or at 3′ end.

In yet another embodiment of the present disclosure, the fluorophore is selected from a group comprising fluorescein and fluorescein derivatives FAM, VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein, tetrachloro-6-carboxyfluoroscein, 5-carboxyrhodamine and cyanine dyes.

In still another embodiment of the present disclosure, quencher is selected from a group comprising Tetra Methyl Rhodamine, 4′-(4-dimethylaminophenylazo) benzoic acid, 4-dimethylaminophenylazophenyl-4′-maleimide, tetramethylrhodamine, carboxytetramethylrhodamine and BHQ dyes.

In still another embodiment of the present disclosure, the preferred Fluorophore is 6-Carboxy Fluorescein [FAM] at 5′ end and the preferred quencher is Tetra Methyl Rhodamine [TAMRA] at 3′ end or black hole quencher 1 (BHQ1) in the internal region or at 3′ end.

The present disclosure relates to primers of SEQ ID Nos. 4 or 10, 5, 6, 7, 8, and 9.

In an embodiment of the present disclosure, the primers having SEQ ID Nos 4 or 10, 5, and 6 are sense primers and the primers having SEQ ID Nos 7, 8, and 9 are anti-sense primers.

In another embodiment of the present disclosure, the primers having SEQ ID Nos 4 or 10 and 7 correspond to Probe of Sequence ID No. 1, primers having SEQ ID Nos 5 and 8 correspond to Probe of Sequence ID No. 2 and primers having SEQ ID Nos 6 and 9 correspond to Probe of Sequence ID No. 3.

The present disclosure relates to a PCR reaction mixture for detection of malaria, said mixture comprising the sample to be detected, nucleic acid amplification reagents, probes selected from a group comprising SEQ ID Nos. 1, 2, and 3 and corresponding primers selected from a group comprising SEQ ID Nos. 4 or 10, 5, 6, 7, 8, and 9.

In an embodiment of the present disclosure, the primers having SEQ ID Nos 4 or 10 and 7 correspond to Probe of Sequence ID No. 1, primers having SEQ ID Nos 5 and 8 correspond to Probe of Sequence ID No. 2 and primers having SEQ ID Nos 6 and 9 correspond to Probe of Sequence ID No. 3.

In another embodiment of the present disclosure, the malarial infection is detected from samples selected from a group comprising blood, saliva and urine sample.

The present disclosure relates to a method of detecting and optionally quantifying malarial infection, said method comprising steps of:
(a) forming a reaction mixture comprising a sample to be detected, nucleic acid amplification reagents, probes selected from a group comprising SEQ ID Nos. 1, 2, and 3 and corresponding primers selected from a group comprising SEQ ID Nos. 4 or 10, 5, 6, 7, 8, and 9;
(b) subjecting the reaction mixture to PCR to obtain copies of target sequence followed by measuring an increase in fluorescence signal for detecting the malarial infection; and
(c) optionally constructing a standard curve from the detected signal to obtain copy number for quantifying the malarial infection.

In an embodiment of the present disclosure, the primers having SEQ ID Nos 4 or 10, 5, and 6 are sense primers and the primers having SEQ ID Nos 7, 8, and 9 are anti-sense primers.

In another embodiment of the present disclosure, the primers having SEQ ID Nos 4 or 10 and 7 correspond to Probe of Sequence ID No. 1, primers having SEQ ID Nos 5 and 8 correspond to Probe of Sequence ID No. 2 and primers having SEQ ID Nos 6 and 9 correspond to Probe of Sequence ID No. 3.

In yet another embodiment of the present disclosure, the fluorescence signal is generated by the probes having fluorophore at the 5′ end along with the quencher in internal region or at 3′ end.

In still another embodiment of the present disclosure, the fluorophore is selected from a group comprising fluorescein and fluorescein derivatives FAM, VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein, tetrachloro-6-carboxyfluoroscein, 5-carboxyrhodamine and cyanine dyes.

In still another embodiment of the present disclosure, the quencher is selected from a group comprising Tetra Methyl Rhodamine, 4′-(4-dimethylaminophenylazo) benzoic acid,4-dimethylaminophenylazophenyl-4′-maleimide,tetramethylrhodamine, carboxytetramethylrhodamine and BHQ dyes.

In still another embodiment of the present disclosure, the malarial infection is detected from samples selected from a group comprising blood, saliva and urine sample.

The present disclosure relates to a kit for detection of malarial infection, said kit comprising dual labeled probes of SEQ ID Nos.1, 2, and 3 individually or in combination; corresponding pair of primers of SEQ ID Nos. 4 or 10, 5, 6, 7, 8, and 9 individually or in combination and amplification reagents.

In an embodiment of the present disclosure, the amplification reagents include magnesium chloride, Taq polymerase and buffer for amplification.

### Regions Chosen for Probe and Primer Designing

Probe having SEQ ID No. 1 along with Primers having SEQ ID Nos. 4 or 10 and 7 were designed for the erythrocyte binding protein region of Plasmodium falciparum. Similarly SEQ ID No. 2 probe along with SEQ ID Nos. 5 and 8 primers were designed for the Var gene regions of Plasmodium falciparum. SEQ ID No.3 probe along with SEQ ID Nos. 6 and 9 primers were designed for erythrocyte binding protein gene of Plasmodium vivax.

The objective of the present disclosure is detection of malarial infection caused by either Plasmodium falciparum or Plasmodium vivax from DNA isolated from infected blood, saliva or urine samples. The mode of detection is by monitoring increase in fluorescence by real time PCR using “Oligonucleotide” probes labeled with a fluorophore and a quencher.

The present disclosure is with regard to the detection of malarial infection using Oligonucleotide probes and their respective primers employing real time PCR method.

The above mentioned “Oligonucleotide” probes are conjugated to a fluorophore at the 5′ end and quencher in the internal region or at the 3′ end.

In still another embodiment of the present disclosure said fluorophore is selected from a group comprising fluorescein and fluorescein derivatives FAM, VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein, tetrachloro-6-carboxyfluoroscein, 5-carboxyrhodamine and cyanine dyes.

In still another embodiment of the present disclosure said quencher is selected from a group comprising Tetra Methyl Rhodamine, 4′-(4-dimethylaminophenylazo) benzoic acid, 4-dimethylaminophenylazophenyl-4′-maleimide, tetramethylrhodamine, carboxytetramethylrhodamine and BHQ dyes.

In another embodiment of the present disclosure said fluorophore is 6-Carboxy Fluorescein [FAM] and the quencher is Black hole quencher 1 [BHQ1] when present internally and Tetra Methyl Rhodamine [TAMRA] or Black hole quencher 1 [BHQ1] when present at the 3′ end.

According to the present disclosure the probes designated by SEQ ID Nos. 1 and 2 are designed for the detection of Plasmodium falciparum. SEQ ID Nos. 4 or 10 and 7 are designed for SEQ ID No. 1 Probe and SEQ ID Nos. 5 and 8 are designed for SEQ ID No. 2 probe respectively.

Similarly the SEQ ID No. 3 probe is designed for the detection of Plasmodium vivax in combination with SEQ ID No. 6 and 9 primers respectively.

According to the present disclosure SEQ ID Nos. 4 or 10, 5 and 6 are sense primers and the SEQ ID Nos. 7, 8 and 9 are anti-sense primers.

The present disclosure is in relation to a method for detecting malarial infection, where in the said PCR mixture comprising of nucleic acid amplification reagents, oligonucleotide probe designated as SEQ ID Nos.1, 2, & 3 with their corresponding primers and malarial DNA sample is subjected for amplification using real-time PCR to obtain copies of the target sequence. The amplification is measured in terms of increase in fluorescence signal and the amount of signal produced is compared with uninfected DNA samples.

According to the present disclosure oligonucleotide probes are having a size ranging from 27-29 nucleotides. The designed probe has a fluorophore at the 5′ end and quencher in the internal region or at the 3′ end.

The fluorophore at the 5′ end is FAM (6-Carboxy Fluorescein) and the quencher is Black hole quencher 1 [BHQ1] when present internally and Tetra Methyl Rhodamine [TAMRA] or Black hole quencher 1 [BHQ1] when present at the 3′ end.

The current disclosure is used for the detection of malarial infection caused by either Plasmodium falciparum or Plasmodium vivax using DNA isolated from blood, urine or saliva samples. The method employed for detection is by using real time PCR.

According to the present disclosure the “Oligonucleotide probe” refers to a short sequence of deoxyribonucleic acid (DNA). The Oligonucleotide probe can specifically hybridise to the target DNA without exhibiting non-specific hydridisation to uninfected DNA.

The probes employed here follow the principles of Taqman chemistry. TaqMan probes also called Double-Dye oligonucleotide or dual labeled probes, are the most widely used type of probes.

The oligonucleotide probe according to the present disclosure is further provided with respective sense and anti-sense primers that can be used to specifically amplify and detect malarial infections caused by either Plasmodium falciparum or Plasmodium vivax by real time PCR. The primers as claimed above have a size ranging from 20-28 nucleotides. The corresponding Probe and Primer sequences for Plasmodium falciparum and Plasmodium vivax are as shown in tables 1, 2, & 3.

TABLE 1

| Sequence name | Nucleotide sequence |
|---|---|
| SEQ ID No. 1 | 5′- FAM- AGTGAACCCCTCACTACCTCTCCTCCAGA- TAMRA- 3′ |
| | Or |
| | 5′- FAM- AGTGAACCCCTCAC/iBHQ1T/ACCTCTCCTCCAGA/3Phos/-3′ |
| | Or |
| | 5′- Flurophore- AGTGAACCCCTCACTACCTCTCCTCCAGA- Quencher- 3′ |
| SEQ ID No. 4 | 5′-TGGAACGAATTCCCTTTTTG-3′ |
| Or | Or |
| SEQ ID No. 10 | 5′-GTCAAATACAAGACCAAGAAACC-3′ |
| SEQ ID No. 7 | 5′-CGCATTTCTCACTTGTTCCA-3′ |

TABLE 2

| Sequence name | Nucleotide sequence |
|---|---|
| SEQ ID No. 2 | 5′- FAM- CCCTCCACAACCAACTGGAACTATTCCA- TAMRA- 3′ |
| | Or |
| | 5′- FAM- CCCTCCACAACCAAC/iBHQ1T/GGAACTATTCCA/3Phos/- 3′ |
| | Or |
| | 5′- Flurophore- CCCTCCACAACCAACTGGAACTATTCCA- Quencher- 3′ |
| SEQ ID No. 5 | 5′-CAAACAAGTATACATAGTGTTGCCAAAC-3′ |
| SEQ ID No. 8 | 5′-TGTTACCACTATGTGTCTCATTTTCC-3′ |

TABLE 3

| Sequence name | Nucleotide sequence |
|---|---|
| SEQ ID No. 3 | 5′-FAM-TTCACCCACTCACCGACTTGACTCAGC- TAMRA- 3′ |
| | Or |
| | 5′-FAM-TTCACCCAC/iBHQ1T/CACCGACTTGACTCAGC/3Phos/- 3′ |
| | Or |
| | 5′- Flurophore- TTCACCCACTCACCGACTTGACTCAGC- Quencher- 3′ |
| SEQ ID No. 6 | 5′-GCGGATTCACGCATCAGTTA-3′ |
| SEQ ID No. 9 | 5′-TATCCGAATGGTAAAGCAAAATAAGTG-3′ |

The oligonucleotide probe according to present disclosure can find application for the detection of malarial infection caused by either Plasmodium falciparum or Plasmodium vivax.

The efficiency of these probes and primers in detecting malarial infections is illustrated by the following examples.

The present disclosure is further elaborated by the following examples and figures. However, these examples should not be construed to limit the scope of the disclosure.

### Example 1

DNA was isolated from a sample panel consisting of 10 blood samples positive for Plasmodium falciparum and 10 uninfected blood samples. Similarly DNA was isolated from 10 blood samples positive for Plasmodium vivax and 10 uninfected blood samples using a commercial DNA isolation kit. The purified DNA was subjected to Real time PCR using SEQ ID No. 1 Probe along with SEQ ID No. 4 or 10 and 7 or SEQ ID No. 2 Probe along with SEQ ID Nos. 5 and 8 for the detection of Plasmodium Falciparum. Similarly SEQ ID No. 3 along with SEQ ID Nos. 6 and 9 was used for the detection of Plasmodium Vivax. Same concentrations of Real time-PCR reagents, template and primers were used in each case and also cycling conditions were kept constant for all the reactions. The composition of PCR mix and PCR conditions are as given in Table 4 & 5.

TABLE 4
Real time-PCR with Takara Premix
Real time PCR Master Mix Composition

| | |
|---|---|
| Premix | 5.0 μl |
| Forward Primer | 0.2 μl(2picomoles) |
| Reverse Primer | 0.2 μl(2picomoles) |
| Probe | 0.2 μl(2picomoles) |
| Sample | 2.0 μl |
| Total | 2.4 μl |

TABLE 5
Real time-PCR cycle conditions
PCR Program

| | |
|---|---|
| Step 1 | 95° C. for 60 sec |
| Step 2 | 95° C. for 5 sec |
| Step 3 | 60° C. for 34 sec |

### Step 2 and 3 are Repeated 40 Times

Results obtained showed that the probes designated as SEQ ID No. 1 and SEQ ID No. 2 which were designed for the detection of Plasmodium falciparum picked up only the infected samples within 40 cycles (positive sample cutoff) showing 100% specificity and sensitivity and did not show any false amplifications for the negative samples (Table 6).

SEQ ID No. 3 which was designed for the detection of Plasmodium vivax also picked up only the infected samples within 40 cycles (positive sample cutoff) showing 100% specificity and sensitivity and it did not show any false amplification for the negative samples (Table 7).

TABLE 6

| | Ct SEQ ID No 1 | Ct SEQ ID No 1 | Ct SEQ |
|---|---|---|---|
| Sample ID | with SEQ ID No. 4 | with SEQ ID No. 10 | ID No 2 |
| Positive 1 | 27 | 26.1 | 25 |
| Positive 2 | 25 | 24.8 | 25 |
| Positive 3 | 28 | 27.6 | 28 |
| Positive 4 | 32 | 29.5 | 30 |
| Positive 5 | 27 | 25.9 | 26 |
| Positive 6 | 24 | 23.7 | 24 |
| Positive 7 | 32 | 29.3 | 29 |
| Positive 8 | 21 | 20.3 | 19 |
| Positive 9 | 27 | 23.5 | 28 |
| Positive 10 | 25 | 23.8 | 26 |

TABLE 7

| Sample ID | Ct SEQ ID No 3 |
|---|---|
| Positive 1 | 25 |
| Positive 2 | 25 |
| Positive 3 | 24 |
| Positive 4 | 23 |
| Positive 5 | 27 |
| Positive 6 | 31 |
| Positive 7 | 34 |
| Positive 8 | 29 |
| Positive 9 | 26 |
| Positive 10 | 30 |

### Example 2

In another study DNA was isolated from a double blind sample panel consisting of 25 infected blood samples. The efficiency of SEQ ID Nos. 1, 2, & 3 in detecting malaria from infected blood samples were then tested by real time PCR. The results obtained were then compared with the other commercial techniques for malaria detection viz, microscopy and rapid diagnostic tests (RDT).

The results obtained showed that SEQ ID Nos. 1, 2 and 3 picked up even the cases of mixed infections which were shown as single infections by the other two techniques. If we look into the Ct values in case of mixed infections the Ct obtained for vivax infections were late and the parasite load at that Ct would be around 3-5 parasites/μl, which will be a very low count. The microscopy and RDT tests cannot detect such a low level of parasitemia and thus the infections reported by these two tests are as single infections. There were few samples which were not detected by the other two tests and the result shown was undetected (Table 8). From this comparison we can conclude that the designed SEQ ID No 1, 2, & 3 along with their respective primers show 100% specificity and sensitivity in detecting malarial infections.

TABLE 8

| | Ct | | | | | |
|---|---|---|---|---|---|---|
| | SEQ ID No 1 | | | | | |
| | With SEQ ID | Ct | Ct | Real time | Microscopy | |
| Sample ID | No 4 | SEQ ID No 2 | SEQ ID No 3 | PCR result | result | RDT result |
| Sample 1 | 27 | 25 | 29 | Mixed | Falciparum | Falciparum |
| Sample 2 | — | — | 25 | Vivax | Vivax | Vivax |
| Sample 3 | 25 | 25 | 31 | Mixed | Falciparum | Falciparum |
| Sample 4 | 28 | 28 | 27 | Mixed | Falciparum | Falciparum |
| Sample 5 | — | — | 25 | Vivax | Vivax | Vivax |
| Sample 6 | 32 | 30 | 27 | Mixed | undetected | undetected |
| Sample 7 | — | — | 24 | Vivax | Vivax | Vivax |
| Sample 8 | — | — | 23 | Vivax | Vivax | Vivax |
| Sample 9 | — | — | 27 | Vivax | Vivax | Vivax |
| Sample10 | 27 | 26 | 29 | Mixed | Falciparum | Falciparum |
| Sample 11 | 24 | 24 | 34 | Mixed | Falciparum | Falciparum |
| Sample 12 | 32 | 29 | 29 | Mixed | Vivax | Falciparum |
| Sample 13 | — | — | 27 | Vivax | Vivax | Vivax |
| Sample 14 | — | — | 23 | Vivax | Vivax | undetected |
| Sample 15 | — | — | 23 | Vivax | Vivax | Vivax |
| Sample 16 | 21 | 19 | 31 | Mixed | Falciparum | Falciparum |
| Sample 17 | 25 | 20 | 34 | Mixed | Falciparum | Falciparum |
| Sample 18 | 23 | 21 | 30 | Mixed | Falciparum | Falciparum |
| Sample 19 | — | — | 23 | Vivax | Vivax | Vivax |
| Sample 20 | — | — | 26 | Vivax | Vivax | Vivax |
| Sample 21 | — | — | 24 | Vivax | Vivax | Vivax |
| Sample 22 | — | — | 27 | Vivax | Vivax | Vivax |
| Sample 23 | 27 | 28 | 31 | Mixed | Falciparum | Falciparum |
| Sample 24 | 25 | 26 | 22 | Mixed | Mixed | Mixed |
| Sample 25 | 25 | 23 | 27 | Mixed | Falciparum | Falciparum |

### Example 3

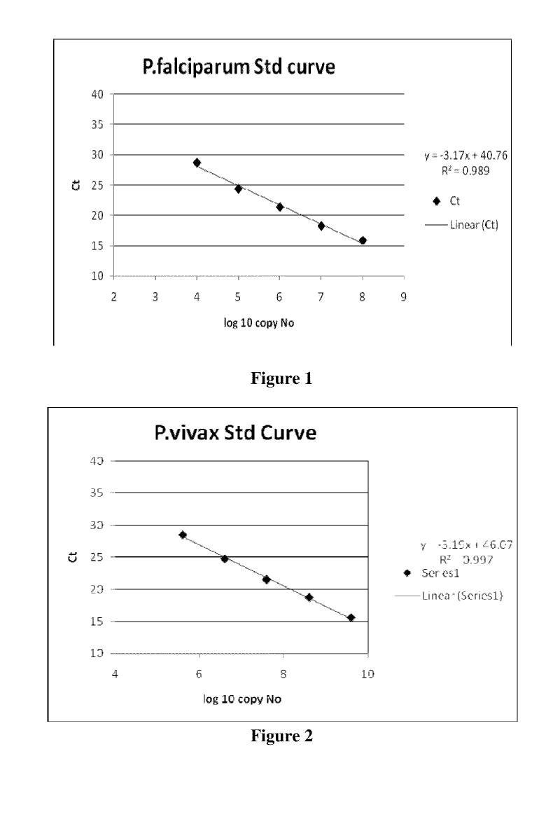

One can also quantify the parasite load from an infected sample by comparing the Ct values obtained from a standard curve (FIGS. 1, 2) & (Tables 9, 10).

### Protocol for Calculation of Copy Number

Around 25 microlitre of malarial DNA (P. Falciparum or P. Vivax) was subjected to PCR along with SEQ ID Nos. 4 or 10 and SEQ ID No.7 primers for P. Falciparum and SEQ ID No.6 and SEQ ID No.9 primers for P. Vivax using a conventional PCR machine. After PCR the amplified samples were run on a agarose gel and stained with ethidium bromide. The amplicon band was then excised from the gel and purified using a Qiaquick gel extraction kit. The absorbance (20 of DNA) was estimated at 260 nm using a nanodrop. Extinction coefficient of the DNA was calculated from individual base coefficient by summing up.

Nanomoles of amplicon was calculated using the following equation:

$$nmoles / ml = \frac{100 \times OD 260 ( 1 cm ) \times 1 ml ( vol )}{Extinction coefficient of amplicon}$$

Copy number was calculated using the formula:

Copy number/ml=(Moles/ml)×Avogadro number.

### Calculations:

### OD 260=0.46

Ext coefficient=3282.12
nmoles/ml=0.14
Copies/ml=8.44×10¹³ for Plasmodium falciparum

### Calculations:

### OD 260=0.182

Ext coefficient=2699.4
nmoles/ml=0.0674
Copies/ml=4.06×10¹³ for Plasmodium vivax

From the copy number of the pure amplicon a standard curve was generated by running 10⁹ to 10⁴ dilutions of the amplicon using a real-time PCR. From the Ct obtained from the standard curve, copy number can be calculated for unknown samples.

Note:—The above mentioned protocol is applicable for the generation of standard curve using SEQ ID No. 5 and SEQ ID No. 8 primers for copy number calculation for var gene region.

TABLE 9
Plasmodium falciparum Copy number with respect to Ct value

| | Cycle | |
|---|---|---|
| Sl no | number (Ct) | Copy no\ml |
| 1 | 20 | 2, 88, 40, 315 |
| 2 | 22 | 64, 26, 877 |
| 3 | 24 | 14, 32, 187 |
| 4 | 26 | 3, 19, 153 |
| 5 | 28 | 71, 121 |
| 6 | 30 | 15, 848 |
| 7 | 31 | 7, 481 |
| 8 | 32 | 3, 531 |
| 9 | 33 | 1, 667 |
| 10 | 34 | 787 |
| 11 | 35 | 371 |

TABLE 10
Plasmodium vivax Copy number with respect to Ct value

| | Cycle | |
|---|---|---|
| Sl no | number (Ct) | Copy no\ml |
| 1 | 20 | 15, 00, 00, 000 |
| 2 | 22 | 3, 60, 00, 000 |
| 3 | 24 | 85, 50, 000 |
| 4 | 26 | 20, 00, 000 |
| 5 | 28 | 4, 83, 000 |
| 6 | 30 | 1, 15, 000 |
| 7 | 31 | 56, 000 |
| 8 | 32 | 27, 000 |
| 9 | 33 | 13, 300 |
| 10 | 34 | 6, 400 |
| 11 | 35 | 3, 160 |

### Example 4

### Malaria Detection from Urine Samples

DNA was isolated from the urine samples of 10 malarial patients positive for Plasmodium falciparum and Plasmodium vivax respectively, using a commercial DNA isolation kit. The purified DNA was subjected to real time PCR using SEQ ID No 1 along with SEQ ID Nos. 4 or 10 and 7 for the detection of Plasmodium Falciparum or SEQ ID No 3 along with SEQ ID Nos. 6 and 9 was used for the detection of Plasmodium Vivax. Same concentrations of Real time-PCR reagents, template and primers were used in each case and also cycling conditions were kept constant for all the reactions.

Results obtained showed that the probes designated as SEQ ID No 1 and SEQ ID No 3 which were designed for the detection of Plasmodium falciparum and Plasmodium vivax picked up all the 10 positive urine samples within 40 cycles (positive sample cutoff) (Table 11).

TABLE 11

| | Ct SEQ ID No. 1 | Ct SEQ ID No. 1 | Ct SEQ |
|---|---|---|---|
| Sample ID | with SEQ ID No 4 | with SEQ ID No 10 | ID No. 3 |
| Positive 1 | 34.3 | 33.5 | 34.2 |
| Positive 2 | 36.2 | 35.2 | 35.1 |
| Positive 3 | 35.3 | 34.0 | 34.8 |
| Positive 4 | 34.0 | 33.2 | 36.3 |
| Positive 5 | 33.8 | 33.0 | 35.17 |
| Positive 6 | 33.3 | 32.1 | 36.1 |
| Positive 7 | 34.4 | 33.5 | 35.4 |
| Positive 8 | 31.2 | 30.4 | 36.6 |
| Positive 9 | 32.7 | 31.8 | 36.2 |
| Positive 10 | 33.8 | 32.6 | 35.5 |

### Example 5

### Malaria detection from Saliva Samples

DNA was isolated from the saliva samples of 5 malarial patients positive for Plasmodium falciparum and Plasmodium vivax respectively using a commercial DNA isolation kit. The purified DNA was subjected to real time PCR using SEQ ID No 1 along with SEQ ID Nos. 4 or 10 and 7 or SEQ ID No. 3 along with SEQ ID Nos. 6 and 9 for the detection of Plasmodium falciparum and Plasmodium vivax respectively. Same concentrations of Real time-PCR reagents, template and primers were used in each case and also cycling conditions were kept constant for all the reactions.

Results obtained showed that the probes designated as SEQ ID No. 1 and SEQ ID No. 3 which were designed for the detection of Plasmodium falciparum and Plasmodium vivax picked up all the 5 positive saliva samples within 40 cycles (positive sample cutoff) (Table 12).

TABLE 12

| | Ct SEQ ID No. 1 | Ct SEQ ID No. 1 | Ct SEQ |
|---|---|---|---|
| Sample ID | with SEQ ID No 4 | with SEQ ID No 10 | ID No. 3 |
| Positive 1 | 38.4 | 37.5 | 34.9 |
| Positive 2 | 34.8 | 33.6 | 39.1 |
| Positive 3 | 34 | 33.2 | 33.1 |
| Positive 4 | 29.2 | 28.2 | 34.4 |
| Positive 5 | 28 | 27.1 | 20.5 |

### CONCLUSION

1) The oligonucleotide probes, SEQ ID No.1, 2 & 3 picked up all the positive samples and they did not show any reactivity to uninfected samples thus showing that they are 100% specific and 100% sensitive.

2) The probes are efficient in picking up the cases of mixed infections which were not detected by microscopy or RDT tests.

3) The probes can can also be used for quantification of parasite load in an infected sample.

4) Finally, the probes, SEQ ID Nos. 1, 2 & 3 along with their respective primers can detect the cases of malarial infections in blood, urine and saliva samples effectively.

1-21. (canceled)

22. Probes having SEQ ID Nos. 1, 2, and 3.

23. The probes as claimed in claim 22, wherein said probes are for detection of malaria; and said probes are conjugated with detectable labels having fluorophore at 5′ end and a quencher in internal region or at 3′ end.

24. The probes as claimed in claim 23, wherein said fluorophore is selected from a group comprising fluorescein and fluorescein derivatives FAM, VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein, tetrachloro-6-carboxyfluoroscein, 5-carboxyrhodamine and cyanine dyes; and said quencher is selected from a group comprising Tetra Methyl Rhodamine, 4′-(4-dimethylaminophenylazo)benzoic acid, 4-dimethylaminophenylazophenyl-4′-maleimide,tetramethylrhodamine, carboxytetramethylrhodamine and BHQ dyes.

25. The probes as claimed in claim 24, wherein the preferred Fluorophore is 6-Carboxy Fluorescein [FAM] at 5′ end and the preferred quencher is Tetra Methyl Rhodamine [TAMRA] at 3′ end or black hole quencher 1 (BHQ1) in the internal region or at 3′ end.

26. Primers of SEQ ID Nos. 4a or 4b, 5, 6, 7, 8, and 9.

27. The primers as claimed in claim 26, wherein the primers having SEQ ID Nos 4a or 4b, 5, and 6 are sense primers and the primers having SEQ ID Nos 7, 8, and 9 are anti-sense primers.

28. The primers as claimed in claim 26, wherein the primers having SEQ ID Nos 4a or 4b and 7 correspond to Probe of Sequence ID No. 1, the primers having SEQ ID Nos 5 and 8 correspond to Probe of Sequence ID No. 2 and the primers having SEQ ID Nos 6 and 9 correspond to Probe of Sequence ID No. 3.

29. A PCR reaction mixture for detection of malaria, said mixture comprising sample to be detected, nucleic acid amplification reagents, probes selected from a group comprising SEQ ID Nos. 1, 2, and 3 and corresponding primers selected from a group comprising SEQ ID Nos. 4a or 4b, 5, 6, 7, 8, and 9.

30. The reaction mixture as claimed in claim 29, wherein the primers having SEQ ID Nos 4a or 4b and 7 correspond to Probe of Sequence ID No. 1, the primers having SEQ ID Nos 5 and 8 correspond to Probe of Sequence ID No. 2 and the primers having SEQ ID Nos 6 and 9 correspond to Probe of Sequence ID No. 3.

31. The reaction mixture as claimed in claim 29, wherein the sample is selected from a group comprising blood, saliva and urine sample.

32. A method of detecting and optionally quantifying malarial infection, said method comprising steps of:
(a) forming a reaction mixture comprising a sample to be detected, nucleic acid amplification reagents, probes selected from a group comprising SEQ ID Nos. 1, 2, and 3 and corresponding primers selected from a group comprising SEQ ID Nos. 4a or 4b, 5, 6, 7, 8, and 9;
(b) subjecting the reaction mixture to PCR to obtain copies of target sequence followed by measuring any increase in fluorescence signal for detecting the malarial infection; and
(c) optionally constructing a standard curve from the detected signal to obtain copy number for quantifying the malarial infection.

33. The method as claimed in claim 32, wherein the primers having SEQ ID Nos 4a or 4b, 5, and 6 are sense primers and the primers having SEQ ID Nos 7, 8, and 9 are anti-sense primers.

34. The method as claimed in claim 32, wherein the primers having SEQ ID Nos 4a or 4b and 7 correspond to Probe of Sequence ID No. 1, the primers having SEQ ID Nos 5 and 8 correspond to Probe of Sequence ID No. 2 and the primers having SEQ ID Nos 6 and 9 correspond to Probe of Sequence ID No. 3.

35. The method as claimed in claim 32, wherein the fluorescence signal is generated by the probes having fluorophore at 5′ end along with the quencher in internal region or at 3′ end.

36. The method as claimed in claim 35, wherein the fluorophore is selected from a group comprising fluorescein and fluorescein derivatives FAM, VIC, JOE, 5-(2′-aminoethyl)aminonaphthalene-1-sulphonic acid, coumarin and coumarin derivatives, lucifer yellow, texas red, tetramethylrhodamine, 6-Carboxy Fluorescein, tetrachloro-6-carboxyfluoroscein, 5-carboxyrhodamine and cyanine dyes; and the quencher is selected from a group comprising Tetra Methyl Rhodamine, 4′-(4-dimethylaminophenylazo)benzoic acid, 4-dimethylaminophenylazophenyl-4′-maleimide,tetramethylrhodamine, carboxytetramethylrhodamine and BHQ dyes.

37. The method as claimed in claim 32, wherein malarial infection is detected from samples selected from a group comprising blood, saliva and urine sample.

38. A kit for detection of malarial infection, said kit comprising dual labeled probes of SEQ ID Nos.1, 2, and 3 individually or in combination; corresponding pair of primers of SEQ ID Nos. 4a or 4b, 5, 6, 7, 8, and 9 individually or in combination and amplification reagents.

39. The method as claimed in claim 38, wherein said amplification reagents include magnesium chloride, Taq polymerase and buffer for amplification.


SEQUENCE LISTING (10 sequences)

SEQ ID NO: 1 - DNA, 29 bp, Plasmodium falciparum
agtgaacccctcactacctctcctccaga

SEQ ID NO: 2 - DNA, 28 bp, Plasmodium falciparum
ccctccacaaccaactggaactattcca

SEQ ID NO: 3 - DNA, 27 bp, Plasmodium vivax
ttcacccactcaccgacttgactcagc

SEQ ID NO: 4 - DNA, 20 bp, Plasmodium falciparum
  primer_bind (1)..(20)
tggaacgaattccctttttg

SEQ ID NO: 5 - DNA, 28 bp, Plasmodium falciparum
  primer_bind (1)..(28)
caaacaagtatacatagtgttgccaaac

SEQ ID NO: 6 - DNA, 20 bp, Plasmodium vivax
  primer_bind (1)..(20)
gcggattcacgcatcagtta

SEQ ID NO: 7 - DNA, 20 bp, Plasmodium falciparum
  primer_bind (1)..(20)
cgcatttctcacttgttcca

SEQ ID NO: 8 - DNA, 26 bp, Plasmodium falciparum
  primer_bind (1)..(26)
tgttaccactatgtgtctcattttcc

SEQ ID NO: 9 - DNA, 27 bp, Plasmodium vivax
  primer_bind (1)..(27)
tatccgaatggtaaagcaaaataagtg

SEQ ID NO: 10 - DNA, 23 bp, Plasmodium falciparum
  primer_bind (1)..(23)
gtcaaatacaagaccaagaaacc